(19) United States
(12) Patent Application Publication
(10) Pub. No.: US 2004/0015159 A1
(43) Pub. Date: Jan. 22, 2004

(54) Methods and apparatus for treating the wall of a blood vessel with electromagnetic energy

(75) Inventors: Charles R. Slater, Fort Lauderdale, FL (US); Thomas A. Gallagher, Stamford, CT (US)

Correspondence Address: Gordon & Jacobson, P.C., 65 Woods End Road, Stamford, CT, 06905, US

(73) Assignee: Syntheon, LLC

(21) Appl. No.: 10/621,943

(22) Filed: Jul. 17, 2003

Related U.S. Application Data
(63) Continuation-in-part of application No. 10/358,523, filed on Feb. 5, 2003.
(63) Continuation-in-part of application No. 09/898,867, filed on Jul. 3, 2001.

(51) Int. Cl. A61B 18/18; A61B 18/20

(52) U.S. Cl. 606/32; 606/33; 606/15

(57) ABSTRACT

The method of the invention is to automatically activate an electromagnetic treating device as it is moved through a blood vessel so that an even amount of energy is dispensed per unit length of the blood vessel. Within reasonable limits, the EM device may be moved either slowly or quickly and still dispense the same amount of energy per unit length of blood vessel. An apparatus for performing the method includes an automatic switching apparatus for use with an EM device whereby activation of the EM device is automatically controlled as the EM device is moved through a blood vessel.

This application is a continuation-in-part of application Ser. No. 10/358,523 filed Feb. 5, 2003 and application Ser. No. 09/898,867, filed Jul. 3, 2001.

### BACKGROUND OF THE INVENTION

1. Field of the Invention

The invention relates to the treatment and correction of blood vessels. More particularly the invention relates to methods and apparatus for automatically delivering treating energy to the interior of a blood vessel in an evenly distributed manner.

2. State of the Art

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sack or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling, and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

Phlebosclerosis, the destruction of venous channels by the injection of treating agents, has been used to treat varicose veins since 1853, when Cassaignae and Ebout used ferric chloride. Sodium salicylate, quinine, urea, and sodium chloride have also been used, but the agent more recently favored is sodium tetradecyl sulfate. In order for phlebosclerosis to be effective, it is necessary to evenly dispense the treating agent throughout the wall of the vein without using toxic levels of the treating agent. This is not particularly difficult for the smaller veins. However, it is quite difficult or nearly impossible in larger veins. When a larger vein is injected with a treating agent, the treating agent is quickly diluted by the substantially larger volume of blood which is not present in smaller veins. The result is that the vein is sclerosed (injured) only in the vicinity of the injection. If the procedure is continued, and the injections are far apart, the vein often assumes a configuration resembling sausage links. The problem cannot be cured by injecting a more potent solution of treating agent, because the treating agent may become toxic at such a concentration.

Until recently, the preferred procedure for treating the great saphenous vein was surgical stripping. This highly invasive procedure involves making a 2.5 cm incision in the groin to expose the saphenofemoral junction, where the great saphenous vein and its branches are doubly ligated en masse with a heavy ligature. The distal portion of the vein is exposed through a 1 cm incision anterior to the medial malleolus, and a flat metal or plastic stripper is introduced to exit in the proximal saphenous vein. The leg is held vertically for 30 seconds to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position for three to four minutes to permit broken vessel ends to retract, constrict, and clot.

After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. No attempt is made to ligate the branches or ends of the veins, since stripping has shown it to be unnecessary. Bleeding is controlled by elevation and pressure for two to four minutes. As many as 40 incisions are made in severe cases, but their small size and transverse direction permit closure with a single suture.

Before closure of the incisions, a rolled towel is rolled repeatedly from the knee to the ankle and from the knee to the groin to express any clots that may have accumulated. The groin incision is approximated with three 5-0 nylon mattress sutures and all other incisions are closed with a single suture.

As can be readily appreciated, the stripping and avulsion-extraction procedures are relatively invasive and require significant anaesthesia. It can therefore be appreciated that it would be desirable to provide an alternative, less invasive procedure which would accomplish the same results as stripping and avulsion-extraction.

The two parent applications listed above describe methods and apparatus for delivering a fluid treating agent to the interior of a blood vessel.

Recently, a number of patents have issued disclosing the treatment of varicose veins with RF energy. Illustrative of these recent patents are: U.S. Pat. No. 6,200,312 entitled “Expandable Vein Ligator Catheter Having Multiple Electrode Leads”; U.S. Pat. No. 6,179,832 entitled “Expandable Catheter Having Two Sets of Electrodes”; U.S. Pat. No. 6,165,172 entitled “Expandable Vein Ligator Catheter and Method of Use”; U.S. Pat. No. 6,152,899 entitled “Expandable Catheter Having Improved Electrode Design, and Method for Applying Energy”; U.S. Pat. No. 6,071,277 entitled “Method and Apparatus for Reducing the Size of a Hollow Anatomical Structure”; U.S. Pat. No. 6,036,687 entitled “Method and Apparatus for Treating Venous Insufficiency”; U.S. Pat. No. 6,033,398 entitled “Method and Apparatus for Treating Venous Insufficiency Using Directionally Applied Energy”; U.S. Pat. No. 6,014,589 entitled “Catheter Having Expandable Electrodes and Adjustable Stent”; U.S. Pat. No. 5,810,847 entitled “Method and Apparatus for Minimally Invasive Treatment of Chronic Venous Insufficiency”; U.S. Pat. No. 5,730,136 entitled “Venous Pump Efficiency Test System And Method”; and U.S. Pat. No. 5,609,598 entitled “Method and Apparatus for Minimally Invasive Treatment of Chronic Venous Insufficiency”. These patents generally disclose a catheter having an electrode tip which is switchably coupled to a source of RF energy. The catheter is positioned within the vein to be treated, and the electrodes on the catheter are moved toward one side of the vein. RF energy is applied to cause localized heating and corresponding shrinkage of the adjacent venous tissue. After treating one section of the vein, the catheter can be repositioned to place the electrodes to treat different sections of the vein. For even and consistent cauterization, RF treatment requires that the practitioner be keenly aware of the procedure time. If RF energy is applied for too long, it can cause undesired burns. If RF energy is not applied long enough, the treatment is ineffective.

In addition to RF treatment, laser treatment has been used with some success. See, e.g. U.S. Pat. No. 6,200,332, entitled “Device and Method for Underskin Laser Treatments”; U.S. Pat. No. 6,197,020, entitled “Laser Apparatus for Subsurface Cutaneous Treatment”; and U.S. Pat. No. 6,096,029, entitled “Laser Method for Subsurface Cutaneous Treatment”. Laser treatment shares the same disadvantage of RF treatment. In particular, as with the RF devices, the practitioner must be very careful as to the intensity and duration of the treatment to assure that the treatment is effective but without causing undesired burns.

EM treatment is also used to treat other blood vessel conditions such as arterial blockages. It would also be advantageous to control the intensity and duration of treatment in these procedures.

### INCORPORATION BY REFERENCE

The disclosures of all of the aforementioned U.S. Patents as well as the disclosures of the two parent applications are hereby incorporated by reference herein.

### SUMMARY OF THE INVENTION

It is therefore an object of the invention to provide methods and apparatus for the minimally invasive treatment of blood vessels.

It is also an object of the invention to provide methods and apparatus for the minimally invasive treatment of blood vessels wherein the wall of the blood vessel is evenly treated.

Another object of the invention is to provide methods and apparatus for the minimally invasive treatment of blood vessels which do not utilize high concentration treating agents.

Yet another object of the invention is to provide methods and apparatus for the minimally invasive treatment of blood vessels which do not require that the practitioner carefully monitor the duration, rate, or progression of treatment.

In accord with these objects which will be discussed in detail below, the method of the invention is to automatically activate an RF, laser, or similar device (hereinafter “electromagnetic” or “EM” device) as it is moved through a blood vessel so that an even amount of energy is dispensed per linear unit of the blood vessel. Within reasonable limits, the EM device may be moved either slowly or quickly and still dispense the same amount of energy per linear unit of blood vessel length.

An apparatus according to the present invention includes an automatic switch for use with an EM device whereby activation of the EM device is automatically controlled as the EM device is moved through a blood vessel.

Several embodiments of a suitable apparatus for performing the method of the invention are disclosed using electromechanical, optical, and magnetic switching.

Additional objects and advantages of the invention will become apparent to those skilled in the art upon reference to the detailed description taken in conjunction with the provided figures.

### BRIEF DESCRIPTION OF THE DRAWINGS

FIG. 1 is a high level schematic view of an apparatus for carrying out the methods of the invention;

FIG. 2 illustrates in part one embodiment of the apparatus of FIG. 1;

FIG. 3 illustrates in part a second embodiment of the apparatus of FIG. 1;

FIG. 4 illustrates in part a third embodiment of the apparatus of FIG. 1;

FIG. 5 illustrates in part a fourth embodiment of the apparatus of FIG. 1;

FIG. 6 illustrates in part a fifth embodiment of the apparatus of FIG. 1;

FIG. 7 illustrates in part a sixth embodiment of the apparatus of FIG. 1;

FIG. 8 is a high level schematic view of an actuator handle incorporating an apparatus for carrying out the methods of the invention;

FIG. 9 is a high level schematic rear end view of the trigger engagement of the actuator of FIG. 8;

FIG. 10 is an enlarged view of a ratchet engagement of the actuator of FIGS. 8 and 9; and

FIG. 11 is a high level schematic view of a seventh embodiment of the invention.

### DETAILED DESCRIPTION OF THE PREFERRED EMBODIMENTS

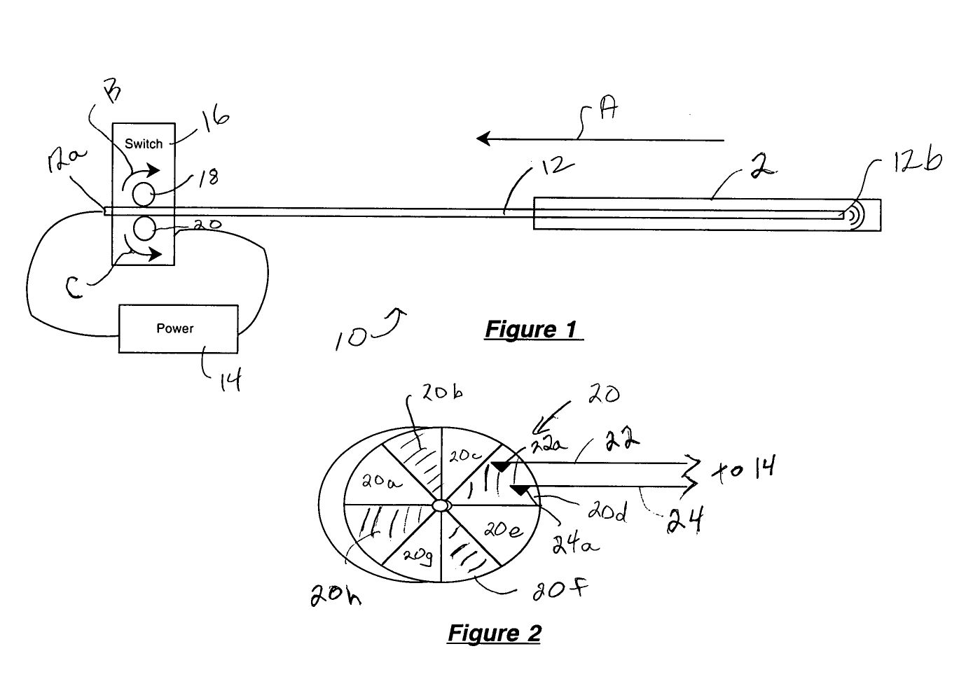

Referring now to FIG. 1, an apparatus 10 for performing the methods of the invention includes an EM device 12, a power supply 14, and a rotary switch 16. The EM device 12 may be any of the prior art electrocautery or laser devices which operate on pulsed power. These devices are in the form of a catheter tube having a proximal end 12a and a distal end 12b. The proximal end 12a is electrically coupled to the power supply 14 and the distal end 12b has either a plurality of electrodes (not shown) or an optical element for emitting laser light. Whereas the prior art devices are coupled to a manually operated switch (not shown) the apparatus of the invention is coupled to an automatically operated switch 16.

According to this embodiment of an apparatus for performing the methods of the invention the automatically operated switch 16 includes a pair of rollers 18, 20 which engage the EM device 12. One of the rollers, e.g. 20, is shown in detail in FIG. 2. A side surface of roller 20 is divided into multiple sectors, e.g. 20a-20h, some of the sectors are conductive (20b, 20d, 20f, and 20h) and the other sectors are not conductive (20a, 20c, 20e, and 20g). A pair of conductors 22, 24 which are coupled to the power supply 14 are provided with wiper contacts 22a, 24a which are arranged to engage the side surface of the roller 20.

The method of operating the apparatus 10 includes deploying the EM device 12 within a blood vessel 2 in the conventional way. The switch 16 is coupled to the device 12, typically near the proximal end 12a. The device 12 is then withdrawn from the blood vessel 2 by pulling it in the direction shown by the arrow “A” in FIG. 1. Those skilled in the art will appreciate that as the device 12 is pulled through the switch 16 in the direction of the arrow “A”, the rollers 18 and 20 will rotate as indicated by the arrows “B” and “C”. As the roller 20 rotates, the wiper contacts 22a, 24a of the conductors 22, 24 will alternately contact conductive sectors and non-conductive sectors on the side surface of the roller 20. Each time the contacts pass over a conductive sector, the EM device will pulse once for a selected duration. The duration of the pulse is controlled by the power supply according to the prior art. The invention automatically dispenses a predetermined number of pulses per unit length of the blood vessel. It will be appreciated that the number of pulses per unit length of blood vessel (NPUL) can be computed using the equation NPUL=ncs/πd, where ncs is the number of conductive sectors and d is the diameter of the roller.

FIG. 3 shows a second embodiment of an automatic switch where one of the rollers 118 has a plurality of spaced apart holes or transparencies 118a-118i. A light source 126 is located on one side of the roller 118 and a light detector 128 is located at the other side. The detector 128 is coupled to a switching circuit 130 which is coupled to the power supply of the EM device. It will be appreciated that as the roller 118 rotates, light from the source 126 is alternately blocked and un-blocked due to the spacing of the holes or transparencies. The switching circuit 130 is designed to produce alternating open and closed connections depending on the presence or absence of light at the detector.

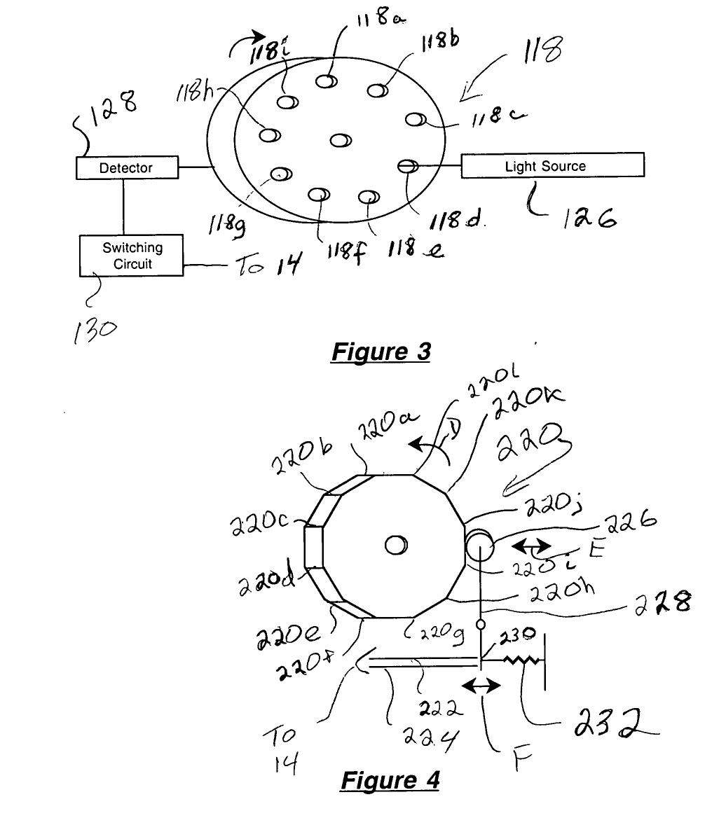

FIG. 4 shows a third embodiment of an automatic switch where one of the rollers 220 has a plurality of spaced apart bumps 220a-220l. A roller 226 is mounted at one end of a lever 228, the other end having an electrically conductive member 230. Conductors 222, 224 are arranged alongside the conductor 230 and are coupled to the power supply of the EM device. The roller 226 is biased against the roller 220 by a spring 232. It will be appreciated that the roller 220 functions as a cam and the roller 226 functions as a cam follower. As the roller 220 rotates as shown by the arrow D, the lever 228 moves back and forth as indicated by the arrow E. This causes the electrically conductive member 230 to move back and forth as shown by the arrow F, making and breaking electrical contact with conductors 222, 224. This causes pulsing of controlled duration and energy per unit length of the blood vessel. Those skilled in the art will appreciate that the roller 220, having a discontinuous surface, may be advantageously arranged so that it does not directly contact the EM device but rather is axially linked to a smooth roller which contacts the EM device and which is rotated by displacement of the EM device.

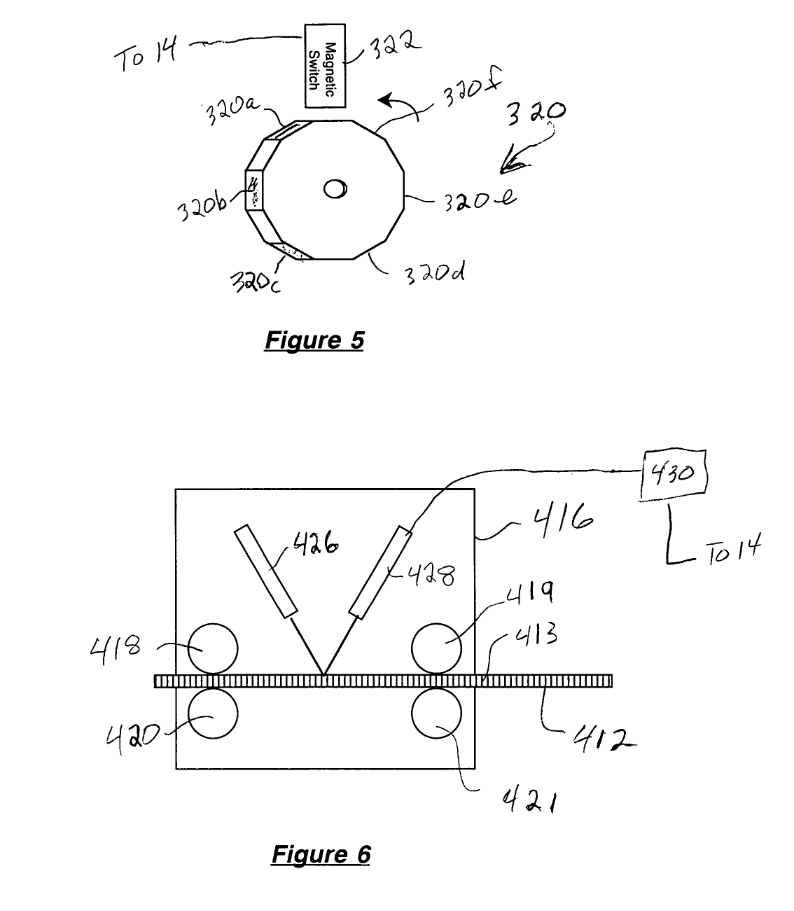

Referring now to FIG. 5, a fourth embodiment of an automatic switch is shown. In this embodiment a roller 320 is provided with a plurality of spaced apart magnets 320a-320f and a magnetically operated switch 322 us located adjacent the roller 320. The switch is coupled to the power supply of the EM device. This causes pulsing of controlled duration and energy per unit length of the blood vessel. Although the roller shown in FIG. 5 has a discontinuous surface like the roller shown in FIG. 4, it will be appreciated that it could be formed with a smooth surface. If it is formed with a discontinuous surface, it may be advantageous to axially couple it to a roller with a smooth surface which contacts the EM device as described above.

FIG. 6 illustrates a fifth embodiment of a switch which, unlike the previous embodiments, does not rely on a roller. The switch 416 shown in FIG. 6 has guides 418, 419, 420, 421 for guiding the EM device 412 through the switch. These guides need not be rollers nor do they need to be round or four in number. It is sufficient that they guide the EM device 412 through the switch. According to this embodiment, the switch is provided with a light source 426 (preferably a laser light source) and a light detector 428. The light detector is electrically coupled to a switching relay 430 which is coupled to the power supply of the EM device. The light source is arranged to direct a beam of light onto the surface of the EM device and the light detector is arranged to detect light reflected off surface of the EM device. The EM device 412 is provided with spaced apart stripes 413 which define areas of light reflectivity and light absorption. Those skilled in the art will appreciate that as the EM device 412 is moved through the switch 416, light from the light source 426 will be alternately reflected and absorbed by the striped surface of the EM device 412 thereby causing pulsing of the EM device. The width and spacing of the stripes determine the number of pulses per unit length of the EM device.

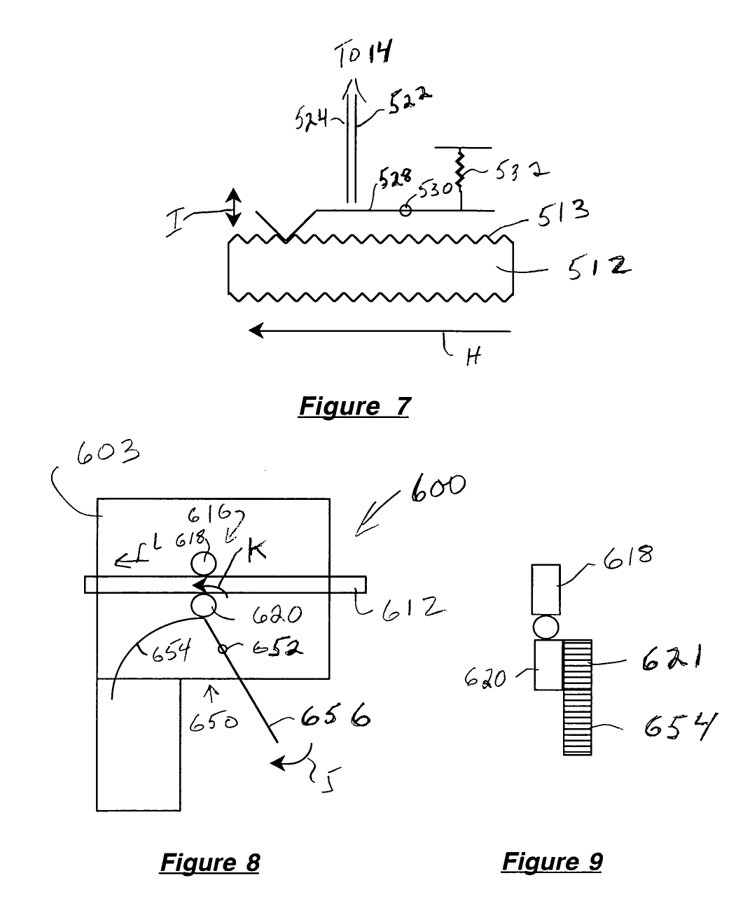

FIG. 7 illustrates a sixth embodiment of an automatic switching arrangement. This embodiment has a pair of electrical contacts 522, 524 arranged adjacent to a movable conductor 528 which is pivotally mounted at 530 and biased away from the contacts by a spring 532. A V-shaped free end 526 extends from the conductor 528. This switch embodiment is intended to be used with a EM device 512 having ridges 513 on its surface. It will therefore be appreciated that when the EM device 512 is moved in the direction shown by the arrow H, the conductor 528 will move up and down in the directions indicated by arrows I making and breaking contact with the electrical contacts 522, 524. This causes pulsing of controlled duration and energy per unit length of the blood vessel.

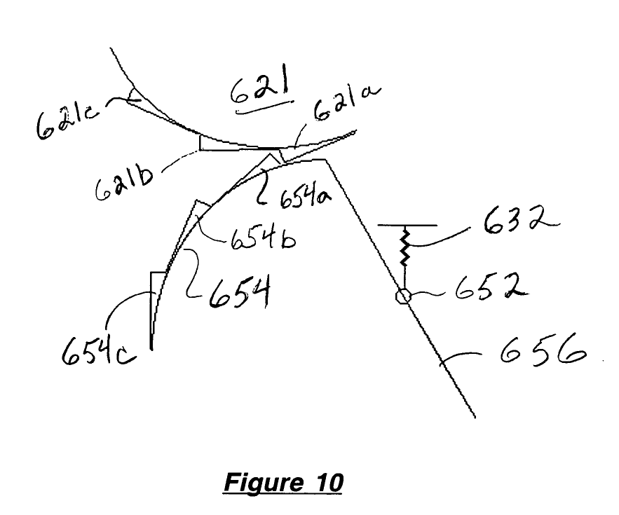

FIGS. 8-10 show an embodiment of an actuator handle 600 for use with an EM device 612. The actuator preferably has a pistol shaped body 603 with a switch 616 coupled to a trigger assembly 650. More particularly, the switch 616 has two rollers 618, 620 which are mounted in the handle 600 and adapted to engage the EM device 612 as described above with reference to the different embodiments of a switch according to the invention. The bottom roller 620 is axially coupled to a ratchet wheel 621 as shown in FIG. 9. The trigger assembly includes a ratchet wheel segment 654 and a trigger 656 which are pivotally coupled to the handle at 652. FIG. 10 shows additional details of the trigger and ratchet assembly which could not be clearly shown in FIG. 8. In particular, it can be seen that the ratchet wheel 621 has a plurality of ratchet teeth, e.g. 621a, 621b, 621c, . . . etc. The ratchet wheel segment 654 is also provided with a plurality of ratchet teeth, e.g. 654a, 654b, 654c, . . . etc. The trigger assembly is spring biased by a spring 632 which urges engagement of the ratchet teeth of the wheel segment 654 with the ratchet teeth of the wheel 621 as shown in FIG. 10.

From the foregoing it will be appreciated that movement of the trigger 656 in the direction of the arrow J in FIG. 8 will cause rotation of the rollers 620 and 621 in the direction of the arrow K which will result in the movement of the EM device 612 in the direction of the arrow L and will actuate the switch 616 as described above resulting in repeatedly pulsing the EM device. Those skilled in the art will appreciate that after the trigger is moved as far as possible in the direction of the arrow J in FIG. 8, it may be returned to the position shown in FIGS. 8 and 10 either manually or by another spring (not shown) and the spring 632 will allow the ratchet teeth of the wheel segment 654 to pass over the ratchet teeth of the wheel 621 without causing the wheel to move.

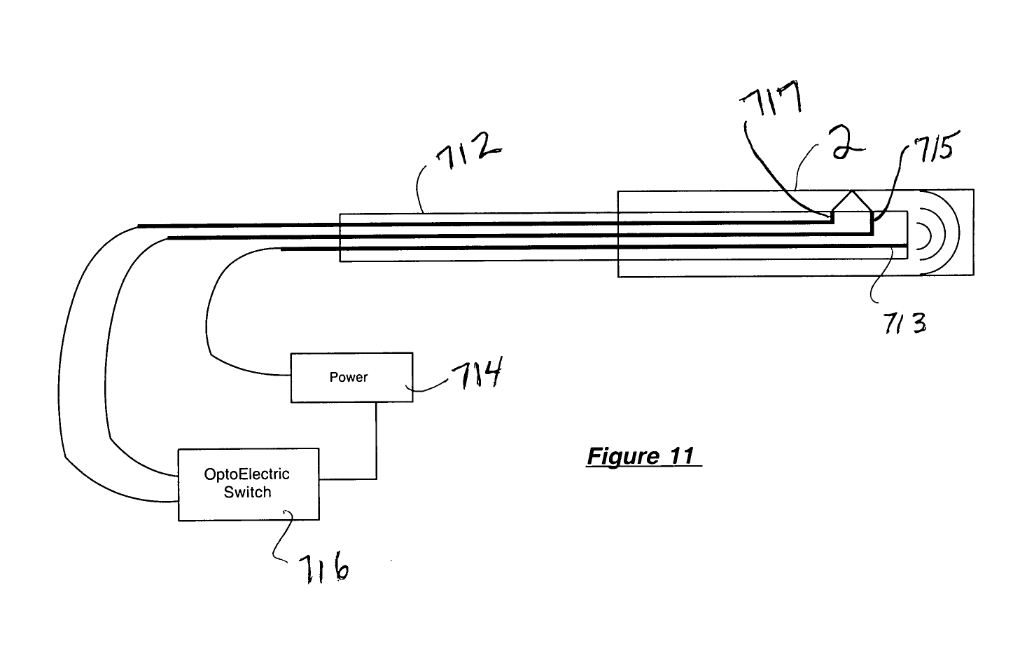

FIG. 11 shows a seventh embodiment of the invention. According to this embodiment, the EM device 712 includes a treating channel 713 (which may be a conductor or a fiber optic depending on the type of device), an optical emission channel 715, and an optical detection channel 717. The optical emission channel 715 emits a light beam toward the inner wall of the blood vessel 2 and the detection channel 717 detects the light reflected off the wall of the blood vessel 2. The emission and detection channels are coupled to optoelectric switching circuits 716 which are coupled to the power supply 714. The optical channels 715, 717 and circuits 716 are configured similar to an “optical mouse” such as disclosed in U.S. Pat. No. 6,501,460 which is incorporated herein by reference. As the device 712 is moved through the blood vessel 2, the optical channels 715, 717 and circuits 716 detect the amount of movement and cause the power supply to pulse the output of the treating channel according to a selected number of pulses per unit length of the blood vessel. It will be appreciated that if the treating channel output is laser light, the wavelength of the emission channel 715 is selected to be distinguishable from the laser light by the detection channel 717.

There have been described and illustrated herein several embodiments of methods and apparatus for treating the wall of a blood vessel using electromagnetic energy (e.g. RF, laser, etc.). While particular embodiments of the invention have been described, it is not intended that the invention be limited thereto, as it is intended that the invention be as broad in scope as the art will allow and that the specification be read likewise. Thus, while particular embodiments of switching apparatus have been disclosed, it will be appreciated that other embodiments could be realized from the teachings of the invention. In other words, it is possible to provide other apparatus which share the common feature of all of the apparatus disclosed, i.e. that the apparatus cause an automatic switching of the EM device as it is moved through a blood vessel so as to provide a set number of pulses per unit length of the blood vessel. Also, while a trigger operated hand held actuator with an automatic switch has been shown, it will be recognized that other types of actuators could be made with automatic switches according to the invention. Further, while the displacement detection means disclosed herein includes rollers, stripes on the EM device co-acting with light detection, and bumps on the surface of the EM device co-acting with a lever, those skilled in the art will appreciate that there are other ways to detect movement of the EM device. It will therefore be appreciated by those skilled in the art that yet other modifications could be made to the provided invention without deviating from its spirit and scope as so claimed.

1. A method for treating the wall of a blood vessel using electromagnetic energy, said method comprising:
a) delivering an EM device into the blood vessel; and
b) translate the EM device through the blood vessel while automatically activating the EM device to deliver a constant amount of energy per unit length of the blood vessel.

2. The method according to claim 1, wherein:
said step of automatically activating the EM device includes automatically pulsing energy.

3. The method according to claim 1, wherein:
said step of automatically activating the EM device includes mechanically detecting movement of the EM device through the blood vessel.

4. The method according to claim 1, wherein:
said step of automatically activating the EM device includes optically detecting movement of the EM device through the blood vessel.

5. The method according to claim 1, wherein:
said step of automatically activating the EM device includes magnetically detecting movement of the EM device through the blood vessel.

6. The method according to claim 1, wherein:
said step of automatically activating the EM device includes electrically detecting movement of the EM device through the blood vessel.

7. An apparatus for treating the wall of a blood vessel using electromagnetic energy, said apparatus comprising:
a) an EM device adapted to be moved through the blood vessel;
b) detection means for detecting movement of the EM device through the blood vessel;
c) a power supply adapted to deliver a pulse of power for a selected duration;
d) switch means coupled to said power supply and said EM device such that activation of said switch means causes said power supply to deliver said pulse of power to said EM device, said switch means also coupled to said detection means such that said switch means is activated by said detection means.

8. The apparatus according to claim 7, wherein:
said detection means is mechanically coupled to said EM device.

9. The apparatus according to claim 8, wherein:
said detection means includes at least one roller.

10. The apparatus according to claim 8 wherein:
said detection means includes a lever.

11. The apparatus according to claim 7, wherein:
said detection means is optically coupled to said EM device.

12. The apparatus according to claim 11, wherein:
said detection means includes a light source and a light detector.

13. The apparatus according to claim 7, wherein:
said switch means is mechanically coupled to said detection means.

14. The apparatus according to claim 13, wherein:
said switch means includes an electrical wiper contact.

15. The apparatus according to claim 13, wherein:
said switch means includes a lever.

16. The apparatus according to claim 7, wherein:
said switch means is optically coupled to said detection means.

17. The apparatus according to claim 7, wherein:
said switch means includes a light source and a light detector with a shutter therebetween.

18. The apparatus according to claim 7, wherein:
said switch means is magnetically coupled to said detection means.

19. The apparatus according to claim 7, further comprising
e) an actuator coupled to said EM device, said actuator being adapted to move said EM device through said blood vessel.

20. The apparatus according to claim 19, wherein:
said actuator includes a manually operated trigger.

21. The apparatus according to claim 7, wherein:
said detection means includes a light source directed at said EM device and a light detector arranged to detect light reflected from said EM device by said light source.

22. The apparatus according to claim 7, wherein:
said detection means includes a light source coupled to said EM device and arranged to direct light toward an interior wall of the blood vessel and a light detector arranged to detect light reflected from the interior wall of the blood vessel by said light source.
